(19) United States
(12) Patent Application Publication
(10) Pub. No.: US 2025/0017514 A1
(43) Pub. Date: Jan. 16, 2025

(54) DIAGNOSTIC DECISION SUPPORT SYSTEM FOR SINGLE AND MULTICHANNEL BPPV CASES THAT WORKS WITH NONPHYSIOLOGICAL FEEDBACK FROM PATIEN

(71) Applicant: Tarik OZKUL, Istanbul (TR)

(72) Inventors: Tarik OZKUL, Istanbul (TR); Murat Haluk OZKUL, Istanbul (TR)

(73) Assignee: Tarik OZKUL, Istanbul (TR)

(21) Appl. No.: 18/711,886

(22) PCT Filed: Nov. 15, 2022
(86) PCT No.: PCT/TR2022/051295

§ 371 Date: May 21, 2024

(30) Foreign Application Priority Data
Nov. 21, 2021 (TR) 2021/018149

(51) Int. Cl. A61B 5/00

(52) U.S. Cl. CPC A61B 5/4023; A61B 5/4884; A61B 5/702; A61B 5/7475

(57) ABSTRACT

Invention is a system that diagnoses the source of the problem in BPPV-induced vertigo patients by applying diagnostic maneuvers within the framework of a dynamic algorithm and getting feedback of voluntary non-physiologic signals from the patient. The system is developed specifically for BPPV patients exhibiting small amplitude nystagmus eye movements or patients exhibiting no nystagmus whatsoever. The system diagnoses BPPV cases by testing different diagnostic positions while getting voluntary feedback signals from the patient. Depending on the feedback received from the patient, the algorithm changes its algorithmic steps and guides the decision process toward a diagnosis. The system is intended to be used with repositioning chairs, as well as manual treatment methods on a stretcher.

### FIELD

The present invention relates to the category of medical devices for investigating source of vestibular disorder called BPPV by seeking nonphysiological feedback from the patient. This is especially important for BPPV cases where patient exhibits weak or no nystagmus signals for diagnostic purposes. More specifically invention relates to a system executing dynamic algorithm guided by voluntary feedback from the patient to diagnose and treat the source of Benign Paroxysmal Positional Vertigo (BPPV) problem.

### BACKGROUND

Benign Paroxysmal Positional Vertigo (BPPV) is a pathology (discomfort) that occurs in the inner ear vestibular organ, which is a part of the body's balance mechanism. This disease causes recurrent vertigo that occurs with head movements. Orientation of patient's head to a specific position triggers the occurrence of vertigo in the patient. Once triggered, vertigo lasts for 30-60 seconds during which patient experiences a rotational hallucination. During the BPPV attack, the patient's balance is disturbed causing nausea, vomiting, cold sweats, etc. During the vertigo attack involuntary rapid eye movements called “nystagmus” occur simultaneously in both eyes of the patient. The nystagmus movements can be quite complicated and contains patterns of movements help identify the source of the canal with problem. This identification is very important for selecting the appropriate treatment type.

The BPPV disease has no known cause. However, its pathophysiology (mechanism) is well understood and it occurs as a result of the calcium carbonate crystals which are fixed in the part of the balance organ called the utricle breaking off and falling into the connected semicircular canals. There are 6 such semicircular canals in the human balance organ. Three of them on the right side and three of them on the left side. The utricle (crystals) line up inside the canal. When these crystals get loose and fall inside the canal, they cause the balance organ to malfunction. It is known that BPPV is caused by loose crystals, but why these crystals get loose is not known. Head trauma, viral diseases, migraine etc. may be the underlying reasons for loose crystals but there is no evidence-based data that establishes the cause of BPPV as of today. None of the imaging methods known in the medical world like MRI or X-Ray help us see the loose crystals.

The only known curing treatment of this ailment is to deposit the loose crystals to a harmless place in the balance organ. This is done by physically orienting the head of the patient where the balance organ located into different positions in 3-dimensional space and to deposit the loose crystals inside the canal into a harmless area of the balance organ where they don't cause malfunction of the balance organ. This needs to be done by a series of movements in 3D space designed specifically for the canal that has loose crystals inside. Since each canal is oriented differently, it is imperative to identify the canal with problem so that appropriate maneuver is executed. Since we cannot see the loose crystals using medical imaging methods, it is very important to diagnose the source of problem canals.

There are a total of 11 types of BPPV treatment maneuvers that need to be selected according to the semicircular canal where the crystals are located. Each of these treatment maneuvers is different, and each maneuver includes an average of five stages. At each stage of the maneuver, the patient's head should be brought to a defined angle at a certain speed.

What complicates the problem even further is the fact that these loose crystals may be in more than one of these semicircular canals. (This is known as multi-canal BPPV.) Since each canal is oriented differently inside the body, each canal requires a different set of treatment maneuvers. It is important to wait a certain period of time between maneuvers at each stage, and not complying with any of these factors is the reason why the treatment maneuver is unsuccessful.

To understand the current state of technology and how the invention contributes to the state of the art, we must first examine the current diagnostic process.

The diagnosis of BPPV is done by bringing the patient into four different “provoking” positions. Each provoking position triggers a BPPV attack specific to a semicircular canal. These are the four different tests that need to be done to understand where the crystals are causing BPPV. Each of these tests is specifically used to detect the presence of crystals in a particular channel. These;
Dix-Hallpike to the right,
Dix-Hallpike to the left,
Roll Right,
Roll Left
known as tests. In order to perform these tests, the patient is put into the specific position mentioned above. These four different diagnostic positions are shown in FIG. 1B. The angular values of these positions and the diagnostic procedure are well known by experts and explained in detail in the reference document (Bhattacharyya et al., 2017).

As the patient is put into these provoking positions, the existence of crystals in the relevant channel causes a trembling movement called “nystagmus” in the patient's eyes. The pattern of the eye movement indicates which semicircular canal the crystals are located in. Additionally, whether the nystagmus movement starts immediately or with a lag, how long it lasts are important factors in the diagnosis. After evaluating all this information, the specialist physician decides what treatment maneuver should be used to reduce the scattered crystals.

Although there are four different diagnostic maneuvers for the diagnosis of the disease, there are 11 different treatment maneuvers to treat the condition. The physician will choose the appropriate treatment maneuvers in the light of information such as the starting time of nystagmus, the direction of the nystagmus movement of the eye, and the duration of nystagmus.

These treatment maneuvers are known by the following names:
1. Right Epley maneuver,
2. Left Epley maneuver,
3. Right Semont maneuver
4. Left Semont maneuver,
5. Right BBQ maneuver,
6. Left BBQ maneuver,
7. Right Gufoni Canalithiasis maneuver,
8. Left Gufoni Canalithiasis maneuver,
9. Right Gufoni Cupulolithiasis maneuver,
10. Left Gufoni Cupulolithiasis maneuver,
11. Deep head hanging maneuver

Although the diagnostic process looks simple and straightforward, in practice there are difficulties that complicate the process. One of the reasons that complicates the problem is the fact that the nystagmus movement is not always easy to observe and decipher. In some cases, the amplitude of nystagmus movement is small and almost not discernable. In some cases, the patient does not exhibit nystagmus at all. In such cases diagnosis process becomes very difficult.

In line with this difficulty, the misdiagnosis rate in BPPV is very high. According to Kerber and Newman-Toker, the misdiagnosis rate of BPPV in the Emergency Room is as high as 74-81%. The difficulties stated above contribute to the extremely high misdiagnosis rate of the BPPV.

Having such a high misdiagnosis rate, the use of AI-based decision support for the diagnosis of BPPV is highly desirable. There have been several approaches to using AI for diagnosis. All of these approaches use analyses of nystagmus data for decision-making. Please see Kabade et al to see the review of existing methodologies of diagnosis.

The invention explained in this application teaches a method of diagnosis that does not rely on nystagmus. The method is different than all other known diagnostic procedures.

### REFERENCES

Kerber K A, Newman-Toker D E. Misdiagnosing Dizzy Patients: Common Pitfalls in Clinical Practice. Neurol Clin. 2015 August; 33 (3): 565-75, viii. doi: 10.1016/j.ncl.2015.04.009. PMID: 26231272; PMCID: PMC9023124.
Neil Bhattacharyya, Samuel P. Gubbels, Seth R. Schwartz, Jonathan A. Edlow, Hussam El-Kashlan, Terry Fife, Janene M. Holmberg, Kathryn Mahoney, Deena B. Hollingsworth, Richard Roberts, Michael D. Seidman, Robert W. Prasaad Steiner, Betty Tsai Do, Courtney C. J. Voelker, Richard W. Waguespack, and Maureen D. Corrigan Clinical Practice Guideline: Benign Paroxysmal Positional Vertigo (Update) Otolaryngology-Head and Neck Surgery 2017, Vol. 156 (3S) S1-S47C American Academy of Otolaryngology-Head and Neck Surgery Foundation 2017 https://doi.org/10.1177/0194599816689667
Lim E C, Park J H, Jeon H J, Kim H J, Lee H J, Song C G, Hong S K. Developing a Diagnostic Decision Support System for Benign Paroxysmal Positional Vertigo Using a Deep-Learning Model. J Clin Med. 2019 May 8; 8 (5): 633. doi: 10.3390/jcm8050633. PMID: 31072056; PMCID: PMC6571642.
Kabade V, Hooda R, Raj C, Awan Z, Young A S, Welgampola M S, Prasad M. Machine Learning Techniques for Differential Diagnosis of Vertigo and Dizziness: A Review. Sensors (Basel). 2021 Nov. 14; 21 (22): 7565. doi: 10.3390/s21227565. PMID: 34833641; PMCID: PMC8621477.

### SUMMARY

The invention aims to diagnose BPPV disease by using voluntary nonphysiological feedback from the patient. What is meant by “diagnosis” should be understood as the identification of semicircular canals with crystals (utricle) inside. Once these semicircular canals with problems are identified, the appropriate treatment maneuver can be selected for the treatment of the patient.

In cases where the nystagmus in patients' eyes is clear and identifiable, the physician can diagnose the BPPV relatively easily. In cases, where the nystagmus of the patient is not visible clearly, all known BPPV diagnostic procedures fail altogether. Regardless of whether the nystagmus visible clearly or not, the patient always experiences dizziness in provoking positions.

The invention makes use of this fact and teaches a method where feedback from the patient is used for diagnostic decision-support purposes. The purpose of the present invention is to make the diagnosis using voluntary feedback from the patient without relying on information from the eyes. In this respect, the invention follows a different approach from all other known decision-support approaches.

Involuntary nystagmus movement and severe dizziness starts as soon as the BPPV episode starts. Even if nystagmus does not appear in the eyes of the patient, the patient feels dizziness with all its severity. The invention aims to indicate the moment and duration of the dizziness by using voluntary signals indicating the starting of dizziness. The voluntary signal can be given by a button pressed or a voice signal. The decision procedure requires some additional information requested from the patient which is provided by the patient providing voluntary signals.

This method also gives very good results in patients with nystagmus clearly visible in their eyes.

The correct interpretation of nystagmus signals constitutes the most difficult and complex part of the diagnosis of BPPV. The invention aims to obtain the most accurate diagnosis through voluntary signals from the patient, regardless of the nystagmus signals from the patient. For this purpose, the patient is asked to press a button as soon as the dizziness starts and keep the button pressed as long as the dizziness continues. During the decision process, at certain decision-making points, the patient is put into different provoking positions and asked which position the dizziness is felt more severely. In general, deciding on which side the dizziness is felt more severely is an easy matter for the patient to decide, and the patient can identify which side he/she feels dizziness more severely without hesitation.

By applying the decision process, the canals with problems are identified and the treatment maneuvers are determined. The process can identify multi-canal BPPV cases as well.

### BRIEF DESCRIPTION OF DRAWINGS

FIG. 1A: Shows the general scheme of the invention

FIG. 1B: Shows four different diagnostic positions that trigger BPPV attack episode

FIG. 2A: Shows parts of the invention

FIG. 2B: Shows the timeline of the interaction of parts of the invention with each other

FIG. 3A: Shows the first part of the diagnosis decision flowchart.

FIG. 3B: Shows the second part of the diagnosis decision flowchart.

### DESCRIPTION

The method of operation of the present invention will be described with the aid of the figures. The invention aims to diagnose BPPV by applying a procedure using voluntary feedback from the BPPV patient. The diagnostic method is designed to diagnose single and multi-canal BPPV cases without relying on the nystagmus of the patient. The purpose of BPPV diagnosis is to find the appropriate treatment maneuver for the patient.

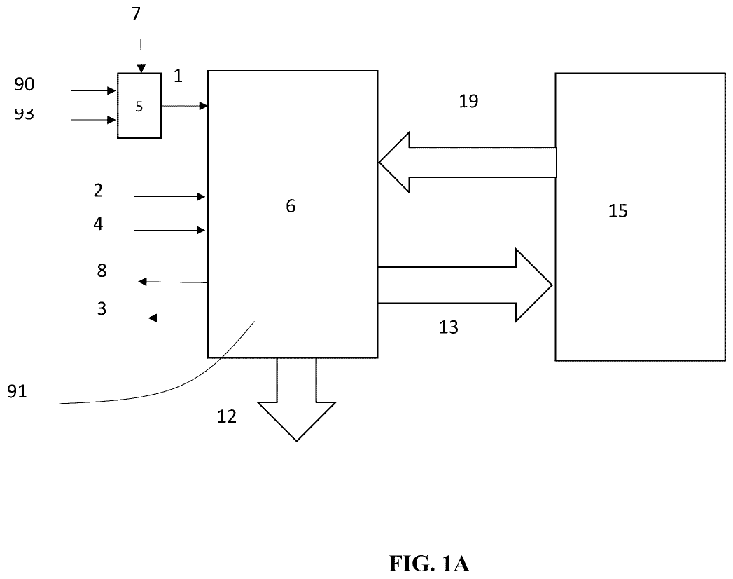

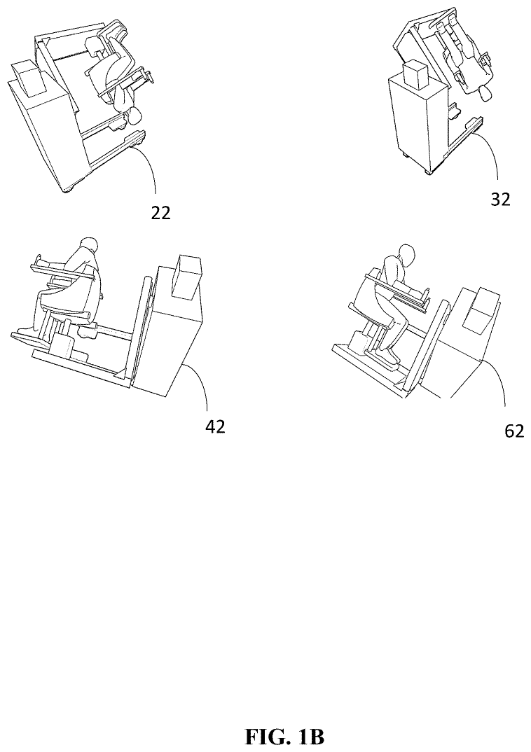

FIG. 1A shows the general scheme of the invention. The system is made up of the Diagnostic decision system (6) and the Voluntary signal generation block (5). There are input signals received and output signals generated by the Diagnostic decision system (6). Patient feedback signals (1) are signals produced by the Voluntary signal generation block (5). The patient gives his/her feedback by using the Patient left button (90), Patient right button (93), and Voluntary audio signal (7). These signals are converted to Patient feedback signals (1) by Voluntary signal generation block (5).

Physician feedback signal right (2) and Physician feedback signal left (4) are the signals given by the physician. The duty of these signals can be explained as follows. During the diagnosis of BPPV, the patient will be put into positions that will trigger the BPPV attack. This is done to figure out which canals have loose crystals inside. During a BPPV attack, the patients are supposed to report the existence of dizziness by pressing the Patient right button (93), Patient left button (90), Voluntary audio signal (7). Most of the patients can follow instructions and use the buttons to signal the start of the BPPV episode. However, in some rare cases, the BPPV attack overwhelms the patient so much that the patient becomes unable to give voluntary feedback signals. In such cases, the attending physician may use the Physician feedback signal right (2) and the Physician feedback signal left (4) to give the signal that the patient should have given. Experienced physicians can tell the start of the BPPV attack by observing the general condition of the patient or looking into the eyes of the patient. If the patient cannot respond, the physician is supposed to signal the start of the BPPV attack by pressing either the Physician feedback signal right (2) or the Physician feedback signal left (4).

Decision algorithm (91) is the algorithm running inside the Diagnostic decision system (6). The Decision algorithm (91) generates some signals that flags the stage of the algorithm.

One of the output signals produced by the Diagnostic decision system (6) is the Relative dizziness severity interrogation phase signal (3). This is an output that notifies the patient that he or she will be placed in two different diagnostic positions in the coming phase and will be asked to report which position causes more severe dizziness. This is a signal that informs the patient for the oncoming process. Relative dizziness severity interrogation phase signal (3) is an output that informs the patient about this stage by using visual and sound means.

Another output signal generated by the Diagnostic decision system (6) is the Signal of query which side caused more severe dizziness (8). Right after the patient is placed in two different diagnostic positions, the Signal of query which side caused more severe dizziness (8) output is activated. This informs the patient that he or she now has to report which of the two diagnostic positions she/he placed previously has caused more severe dizziness. The patient sends his/her response by pressing the Patient right button (93) or the Patient left button (90). The response of the patient changes the flow of the Decision algorithm (91) and helps make the diagnostic decision at the end.

Diagnostic decision system (6) is interfaced to Patient repositioning system (15) through some input and output signals. Patient repositioning system (15) shown in FIG. 1A is the repositioning system that puts the patient into diagnostic positions that will provoke BPPV-induced dizziness. The Patient repositioning system (15) puts the patient into the selected diagnostic position on the command of the Diagnostic decision system (6). The Diagnostic decision system (6) sends the position commands to the Patient repositioning system (15) via the Diagnostic position information (13). Upon receiving the Diagnostic position information (13), the Patient repositioning system (15) physically puts the patient into the designated diagnostic position and sends Patient diagnosis reposition confirmation signal (19) to the Diagnostic decision system (6).

FIG. 1B shows the diagnostic positions that provoke the BPPV attack. These are the positions sent by the Diagnostic decision system (6) to Patient repositioning system (15) through Diagnostic position information (13) output. These positions are four, known as DH Right diagnostic test position (22), DH Left diagnostic test position (32), Roll Right diagnostic test position (42), and Roll Left diagnostic test position (62). These positions are known as BPPV provoking positions and they are used for identifying the balance organ canals with crystals inside. The angular values describing these positions are well known in the medical circles and reported in a consensus document titled “BPPV Clinical Practice Guidelines” written by Bhattacharyya et. al.

Patient repositioning system (15) works under the command of Diagnostic decision system (6). A handshake process between the two is required for healthy diagnosis of BPPV. The handshake process is started by Diagnostic decision system (6) sending the desired diagnostic test position to the Patient repositioning system (15) via Diagnostic position information (13) signal. Depending on the selected diagnosis position, it takes 5-15 seconds for the Patient repositioning system (15) to go to the desired diagnostic test position. In order for the diagnosis to be made properly, the patient must notify the Patient repositioning system (15) when it reaches the desired diagnosis position. This is very important for the correct diagnosis procedure. As soon as the Patient repositioning system (15) reaches the desired diagnostic position, it notifies this information to the Diagnostic decision system (6) via the Patient diagnosis reposition confirmation signal (19).

There are patient repositioning systems available on the market that operate automatically or manually. Robotic repositioning systems can be interfaced to the invention to perform the handshake procedure automatically. In case of manual repositioning systems, the handshake signals and the repositioning maneuvers has to be provided by the attending operator manually.

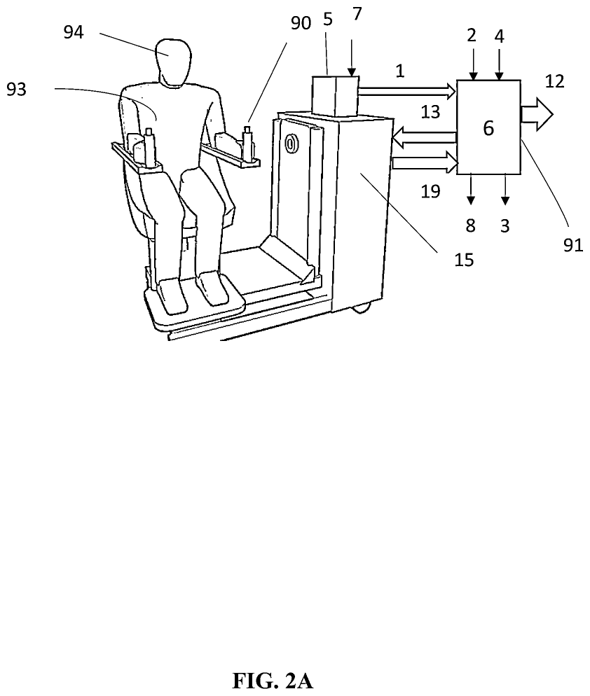

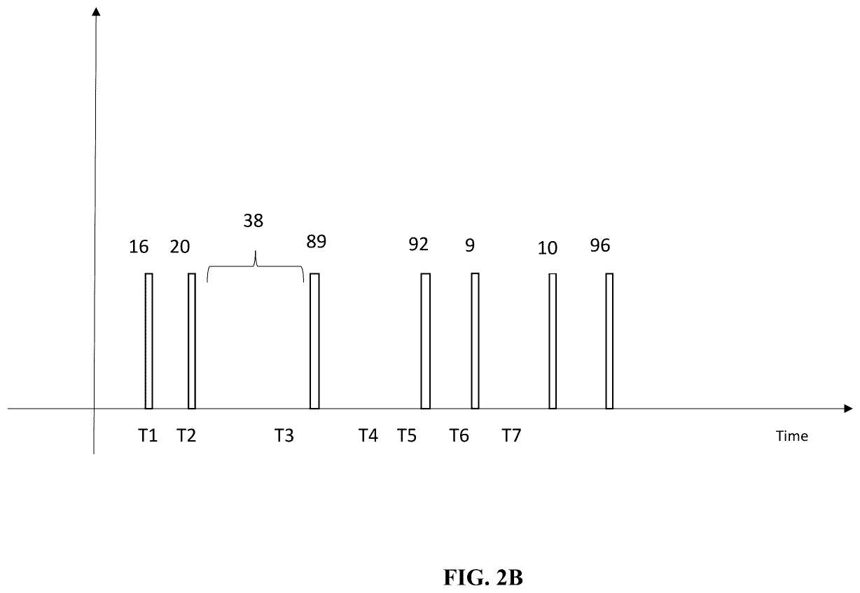

FIG. 2A and FIG. 2B show the parts and working mechanisms of the invention. The Diagnostic decision system (6) gives the command to bring the patient (94) to a diagnostic position within the framework of the algorithm. This information is given via Diagnostic position information (13) to the Patient repositioning system (15). This diagnostic position can be the DH Right diagnostic position (22), or the DH Left diagnostic position (32), or the Roll Right diagnostic test position (42), or the Roll Left diagnostic test position (62). Placing the patient in the diagnostic position triggers BPPV-induced dizziness in the patient. The patient (94) reports the starting instant of dizziness by pressing the Patient right button (93) or the Patient left button (90) or both buttons together. At this stage which button is pressed is not important and the patient (94) can press any button to report the onset of dizziness. The patient (94) is asked to keep pressing the Patient right button (93) or the Patient left button (90) as long as the dizziness continues. How long the dizziness lasts, the moment of onset of the dizziness are important factors for the diagnostic decision. At this stage, the patient (94) can press any button, it doesn't matter if he/she presses the right or left button, but it is important that he/she keeps the button pressed for the duration of the dizziness. Once the patient is placed in one of the diagnostic positions, the dizziness may or may not start right away. Whether the dizziness start right away or start with a delay are important factors for the diagnosis of BPPV. The duration of the dizziness and the moment dizziness start are important pieces of information, and the information will come from the button pressed.

At some stage during the execution of the Decision algorithm (91), the patient (94) will be notified that he/she will be placed in two different provoking positions and will be requested to report which position the dizziness felt more severely. This process is extremely important for the diagnosis, and the patient's feedback will affect the diagnosis. The Diagnostic decision system provides several outputs to indicate the onset of this stage. One of these signals is Relative dizziness severity interrogation phase signal (3). This signal informs the patient (94) to be ready for the coming interrogation. In one embodiment of the technique, the relative dizziness intensity interrogation phase signal (3) is given in the form of an illuminated visual warning. In another embodiment of the technique, the relative dizziness severity interrogation phase signal (3) is given as an audible signal. In another embodiment it may be both visual and audible signals combined. This is a signal for the patient to get mentally ready for the coming process. Another signal used in the process is the Signal of query which side caused more severe dizziness (8) signal. This signal asks the patient (94) to make a selection of which maneuver has caused more severe dizziness by pressing the appropriate button. At this stage, the patient (94) responds to which side caused more dizziness by using the Patient right button (93) or the Patient left button (90). Diagnostic decision system (6) gives the diagnosis of the Patient (94) through the Diagnosis output (12) when all procedures are completed. The diagnosis will lead to the recommended treatment maneuver for the patient's treatment.

The timing of the signals mentioned in FIG. 1A and FIG. 2A are shown in FIG. 2B. The Diagnostic decision system (6) gives the Diagnostic position information (13) to the Patient repositioning system (15). The moment of giving patient diagnosis reposition information (16) is marked on the time axis as T1. When the Patient repositioning system (15) reaches the desired position, it gives the Patient diagnosis reposition confirmation signal (19). This moment is shown in FIG. 2B as (20). The moment of the patient diagnosis reposition confirmation signal (20) and is marked as T2 in the time axis.

The patient (94) is asked to report the moment when the dizziness starts by pressing the Patient right button (93) or the Patient left button (90) and keep it pressed as long as the dizziness lasts. The time lapse between The moment of the patient diagnosis reposition confirmation signal (20) and the patient (94) reporting dizziness by pressing button is called Latency (38) in BPPV terminology. This is a critical piece of information that affects the diagnosis. In FIG. 2B, the patient button press moment (89) shows moment the Patient (94) presses button. This moment is shown in FIG. 2B as T3. The patient (94) is asked to release the button as soon as the dizziness ends. In FIG. 2B, the Patient button release moment (92) is marked as T4 in the time scale. Latency (38) is the time elapsed between the moment of the patient diagnosis reposition confirmation signal (20) and Patient button press moment (89). The duration of the dizziness is measured by a parameter called Duration (39). Duration (39) is the time elapsed between the moment of Patient button press moment (89) and the Patient button release moment (92). Although both Latency (38) and Duration (39) parameters are important, Latency (38) data is more important in the decision process. From a medical point of view, if the Latency (38) time is less than 2 seconds, the patient's Latency (38) is perceived as “no latency”. If the latency (38) is longer than 2 seconds, it is perceived as “latency exists”.

At a certain stage of the decision algorithm, the diagnostic decision system (6) outputs the Relative dizziness severity interrogation phase signal (3). The timing of this signal is shown in FIG. 2B as the Relative vertigo severity questioning phase moment (9) and is marked as T5 on the time axis. After the patient (94) is placed in two different positions, the patient (94) is asked which side caused the more severe dizziness via the output, Signal of query which side caused more severe dizziness (8). The patient (94) answers this question by pressing the Patient right button (93) or the patient left button (90). This moment is shown in FIG. 2B as the Relative dizziness severity response moment (96) and is marked as T7 on the time axis.

The latency and duration are defined as follows:
Latency (38)=T3−T2,
Duration (39)=T4−T3,
No latency: 0<Latency (38)<2 seconds
Latency exists: Latency (38)>2 seconds

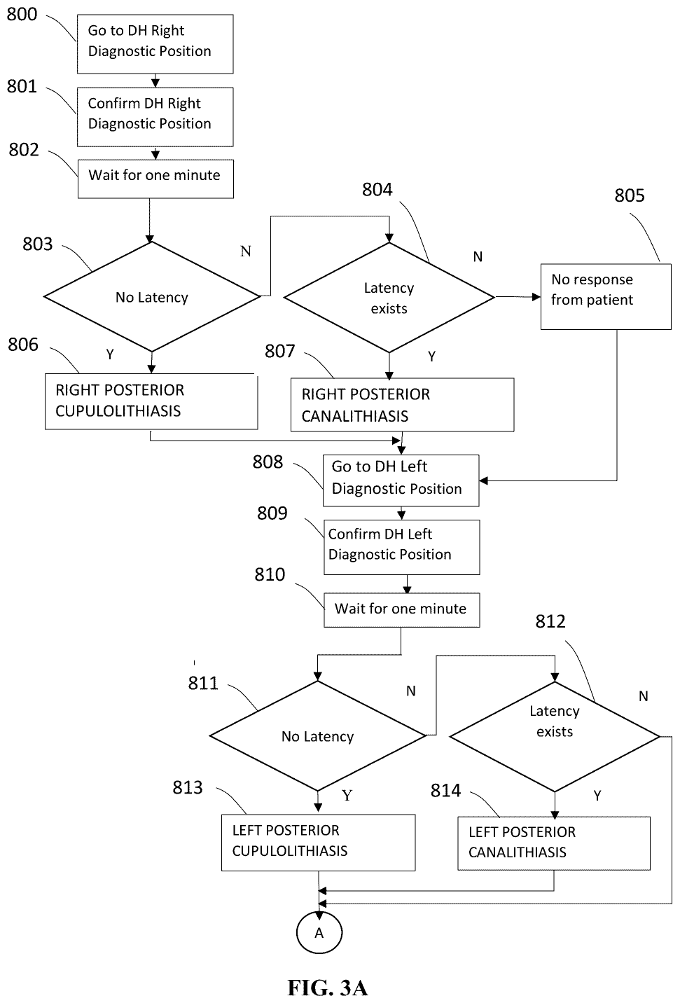

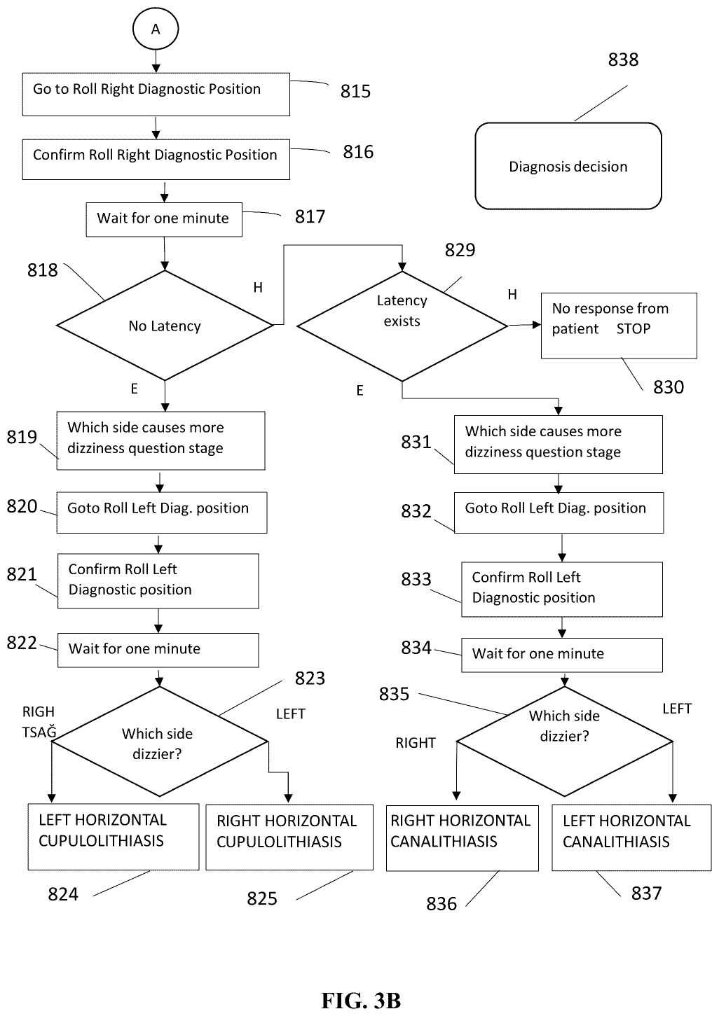

The operation of the Decision algorithm (91) will be explained using FIGS. 3A and 3B. Some parameters described in FIG. 2B will be used to explain the operation of the Decision algorithm (91). The decision algorithm (91) starts with Go to DH Right Diagnostic Position (800) command. DH Right diagnostic test position (22) is a position well known to those who are experts in BPPV and will not be detailed here. The Patient repositioning system (15) then places the Patient (94) in this BPPV provoking position, known as the DH Right diagnostic test position (22). When the Patient repositioning system (15) reaches the desired position it confirms the position, which is shown as the Confirm DH Right Diagnostic Position (801) block. The Patient repositioning system (15) does this confirmation via the Patient diagnosis reposition confirmation signal (19) shown in FIG. 2A. After that, the Decision Algorithm (91) waits for 60 seconds for feedback from the patient (94) in the Wait for one minute (802) block. During this time interval, Patient Feedback Signals (1) are expected from the Patient (94) as to whether or not the dizziness has started. During this period, either the patient does not feel dizzy at all, or the dizziness begins immediately or the dizziness begins with a delay. Technically, the duration of the dizziness is expressed by the Duration (39) parameter, and the delay in the onset of the dizziness is expressed by the Latency (38) parameter. If within the Wait for one minute (802) block, Patient (94) gives Patient Feedback Signals (1) indicating the onset of dizziness in less than 2 seconds, this is considered as No Latency (803). In this case, the diagnosis is Right Posterior Cupulolithiasis (806). Physicians who are experts in the subject know the treatment method needs to be applied for this diagnosis.

If the patient (94) gives Patient Feedback Signals (1) after the first 2 seconds indicating the onset of dizziness, this is considered as Latency exists (804) condition. In this case, the diagnosis will be: Right Posterior Canalithiasis (807). The absence of any dizziness in the patient is detected as No response from patient (805). In this case the Decision Algorithm (91) commands the Patient repositioning system (15) to go to DH Left Diagnostic Position (808). Decision Algorithm (91) commands the Patient repositioning system (15) to Go to DH Left Diagnosis Position (808), regardless the diagnosis was Right Posterior Canalithiasis (807) or Right Posterior Cupulolithiasis (806). This is because the patient may have disease in more than one canal as in the multi-canal cases.

The Patient repositioning system (15) moves to the desired position and confirms reaching the position in Confirm DH Left Diagnostic Position (809) block. After that it waits for one minute in the Wait for one minute block (810). If the Patient (94) gives Patient Feedback Signals (1) during the Wait for one minute (810) block, indicating the onset of dizziness in less than 2 seconds, this is considered as No Latency (811). In this case, the diagnosis will be Left Posterior Cupulolithiasis (813).

If the patient (94) gives Patient Feedback Signals (1) after the first 2 seconds indicating the onset of dizziness, this is considered as latency present in latency exists (812) block. In this case, the diagnosis will be Left Posterior Canalithiasis (814).

The rest of the algorithm is shown in FIG. 3B.

The Decision algorithm (91) then commands the Patient repositioning system (15) to Go to Roll Right Diagnostic Position (815). When the Patient repositioning system (15) reaches the specified position, it gives a confirmation signal shown in Confirm Roll Right Diagnostic Position (816) block. After that, it stays in this position for a minute in Wait for one minute (817) block.

If within the Wait for one minute (817) block, the Patient (94) gives Patient Feedback Signals (1) indicating the onset of dizziness in less than 2 seconds, this is considered as No Latency (818). In this case, the patient (94) is informed that he/she has reached the Which side causes more dizziness question stage (819). Now the patient will be put into another BPPV provoking position and will be asked which one of the positions causes more dizziness. This stage is signaled to the patient (94) by the Relative dizziness severity interrogation phase signal (3) shown in FIG. 2A.

The Decision Algorithm (91) then commands the Patient repositioning system (15) to Go to Roll Left Diagnostic position (820). When the Patient repositioning system (15) reaches the specified position, it gives a confirmation signal in Confirm Roll Left Diagnostic position (821) block. After that, it stays in this position for a minute in Wait for one minute (822) block. The patient (94) is then asked the question Which side is dizzier? (823). This is signaled to the patient (94) by using the Signal of query which side caused more severe dizziness (8) signal shown in FIG. 2A. If the dizziness felt when turning to the right side is more severe, the patient (94) reports this by pressing the Patient right button (93). In this case, the diagnosis will be Left Horizontal Cupulolithiasis (824). If the dizziness felt when the patient (94) turns to the left side is more severe, patient reports this by pressing the Patient left button (90). In this case, the diagnosis will be Right Horizontal Cupulolithiasis (825).

Going back to the Wait for one minute (817) block, if the Patient (94) gives Patient Feedback Signals (1), indicating the onset of dizziness after the first 2 seconds, this is considered as latency exists (829) case. In this case, the patient (94) is informed that he/she has reached the Which side causes more dizziness question stage (831). Now the patient is asked to be prepared for the oncoming decision. The Decision algorithm (91) then commands the Patient repositioning system (15) to Go to Roll Left Diagnostic position (832). When the Patient repositioning system (15) reaches the specified position, it gives a confirmation signal in Confirm Roll Left Diagnostic position (833) block. After that, it stays in this position for one minute (834). After that, the Patient (94) is asked Which side dizzier? (835) question. If the patient (94) feels dizziness was more severe when turning to the right side, he is asked to report this by pressing the Patient right button (93). In this case, the diagnosis will be Right Horizontal Canalithiasis (836).

If the dizziness felt when the patient (94) turns to the left side is more severe, he/she is asked to report this by pressing the Patient left button (90). In this case, the diagnosis will be Left Horizontal Canalithiasis (837).

Wait for one minute (817) block waits for a response from the patient (94). If the Patient (94) does not feel any dizziness during this time frame, it is perceived as No response from the Patient STOP (830) case and at this point the test ends.

Diagnostic decision blocks (824), (825), (836), (837) and (830) are terminal blocks where the decision algorithm (91) stops. Upon reaching this stage, diagnosis decision (838) is reached and findings are reported via the Diagnosis output (12) on the Diagnostic decision system (6). In one of the embodiments of the invention the Diagnosis output (12) may be in the form of a data displayed on a visual display. In another embodiment it may be in the form of a printout or message.

What is claimed is:

1. A method designed to diagnose BPPV condition of vertigo patients based on voluntary signals given by the patient (94) within a decision algorithm (91) by; positioning patient (94) to DH Right diagnostic test position (22), DH Left diagnostic test position (32), Roll Right diagnostic test position (42) and Roll Left diagnostic test position (62), calculating Latency (38), Duration (39) parameters by using Patient feedback signals (1), interrogating the patient (94) which position causes more dizziness in Which side dizzier (823) questioning, interrogating the patient (94) which position causes more dizziness in Which side dizzier (835) questioning, by getting feedback of the patient (94) by using Patient feedback signals (1), reaching diagnosis decision (838) and generating diagnosis output (12).

2. A method of claim 1, where Latency (38) is found as the duration between the moment of the patient diagnosis reposition confirmation signal (20) and the patient button press moment (89).

3. A method of claim 1, where Duration (39) is found as the duration between the patient button press moment (89) and Patient button release moment (92).

4. A method of claim 1, where which side dizzier (823), (835) questioning is responded by patient feedback signals (1) given by the patient (94).

5. A method of claim 1 where latency (38) duration between 0 and 2 seconds it is considered as no latency (803).

6. A method of claim 1 where latency (38) duration more than 2 seconds it is considered as latency exists (804).

7. A method of claim 4 where diagnosis decision (838) is made by positioning the patient (94) in Roll Right diagnostic test position (42) and Roll Left diagnostic test position (62), asking the patient (94) to reply the moment of questioning which side is more violent (10) and the patient (94) giving feedback using patient feedback signals (1).

8. An apparatus designed to diagnose the BPPV problem of vertigo patients comprising;
diagnostic decision system (6),
patient feedback signals (1),
voluntary signal generation block (5),
patient right button (93),
patient left button (90),
physician feedback signal right (2),
physician feedback signal left (4),
diagnostic position information (13),
signal of query which side caused more severe dizziness (8),
relative dizziness severity interrogation phase signal (3),
diagnosis output (12),
and decision algorithm (91).

9. An apparatus of claim 8 where patient feedback signals (1) is given by the patient (94) by pressing patient right button (93) and patient left button (90).

10. An apparatus of claim 8 where the patient (94) is unable to give his/her feedback, patient feedback signals (1) is given by the attending assistant by pressing physician feedback signal right (2) and physician feedback signal left (4).

11. An apparatus of claim 8 where patient feedback signals (1) is generated by the voluntary signal generation block (5) by voluntary audio signal (7) given by the patient (94).

12. An apparatus of claim 8 where diagnostic decision system (6) commands the Patient repositioning system (15) to go to DH Right diagnostic test position (22), DH Left diagnostic test position (32), Roll Right diagnostic test position (42), Roll Left diagnostic test position (62) by giving diagnostic position information (13).

13. An apparatus of claim 8 where diagnostic decision system (6) generates latency (38) information based on the time difference of patient diagnosis reposition confirmation signal (19) given by patient repositioning system (15) and patient button press moment (89) given by the patient (94).

14. A method designed to diagnose BPPV cases by way of voluntary signals given by the patient (94) where decision algorithm (91) changes the flow of the algorithm based on positioning the patient (94) to Roll Right diagnostic test position (42) and Roll Left diagnostic test position (62) consecutively and asking the patient (94) to reply which side dizzier (823) question using the patient feedback signals (1) and the latency (38) information.
